(19) United States
(12) Patent Application Publication
(10) Pub. No.: US 2026/0196359 A1
(43) Pub. Date: Jul. 9, 2026

(54) INTERACTIVE MEDICAL GUIDELINE METHODS AND SYSTEMS

(71) Applicant: Roche Molecular Systems, Inc., Pleasanton, CA (US)

(72) Inventors: Karina KORNOBIS, Slupsk (PL); Richard LAM, Rancho Cordova, CA (US); Ernest LO, Piedmont, CA (US); Marc Philip MAILMAN, Hayward, CA (US); Cezary NOWAKOWSKI, Abramow (PL); Katarzyna ROQUAIN-KUCHARSKA, Warsaw (PL); Vishakha SHARMA, Foster City, CA (US); Pawel WOJTKOW, Warsaw (PL)

(21) Appl. No.: 19/551,108

(22) Filed: Feb. 26, 2026

Related U.S. Application Data
(63) Continuation of application No. PCT/US2024/044386, filed on Aug. 29, 2024.
(60) Provisional application No. 63/535,612, filed on Aug. 31, 2023.

(51) Int. Cl. G16H 70/20; G06F 3/0482; G06F 3/04845; G16H 10/60

(52) U.S. Cl. CPC G16H 70/20; G06F 3/0482; G06F 3/04845; G16H 10/60; G06F 2203/04806

(57) ABSTRACT

A navigable directed graph interface representing a medical guideline, wherein the medical guideline includes a decision tree including a plurality of clinical decisions and preconditions. The navigation interface presents a portion of the directed graph based on a sequence of recorded preconditions and/or clinical decisions of the medical guideline and a portion of the directed graph diverging from the sequence of recorded preconditions and/or clinical decisions. The diverging portion of the directed graph is graphically distinguished from the portion of the directed graph based on the recorded preconditions and/or clinical decisions. In response to receiving a selection of an alternate node from the diverging portion of the directed graph, the graphical representation of the directed graph displays one or more child nodes of the selected alternate node.

### CROSS-REFERENCE TO RELATED APPLICATIONS

This application is a US bypass continuation of International Application No. PCT/US2024/044386, filed on Aug. 29, 2024, which claims the benefit of and priority to U.S. Provisional Application No. 63/535,612, filed on Aug. 31, 2023, the entire disclosures of which are hereby incorporated herein by reference in their entireties for all purposes.

### BACKGROUND

A medical guideline generally refers to a document with the aim of guiding decisions and criteria regarding diagnosis, management, and treatment in specific areas of healthcare. The medical guideline may provide the most current data about prevention, diagnosis, prognosis, therapy including dosage of medications, risk/benefit and cost-effectiveness of a treatment for a particular disease. The guideline may also identify available/decision/treatment options and their outcomes at a particular stage of the disease or the treatment, and different options/outcomes at each can be identified at different stages of the disease/treatment. Based on a current medical condition of a patient, a clinician can refer to the guideline to obtain the different treatment options and possible outcomes, and can determine a treatment option for the patient.

Given that a medical guideline identifies the many available decision/treatment options and their outcomes at different stages of the disease/treatment, the medical guideline is typically a large document having hundreds, if not thousands, of pages. For example, the National Comprehensive Cancer Network® (NCCN) Clinical Practical Guidelines in Oncology for Breast Cancer has more than 200 pages. It can be difficult for a clinician to efficiently navigate through such large documents and identify, compare, and/or select from the different treatment options/outcomes for a given medical condition and patient.

### BRIEF SUMMARY

Disclosed herein are techniques for providing an interactive medical guideline engine. The interactive medical guideline engine includes a navigation interface to provide a graphical representation of a medical guideline to a user, such as a medical care provider (e.g., a clinician, a clinician staff member, etc.). The medical guideline may include a decision tree, which includes a set of clinical decisions, such as a treatment, a diagnosis, etc., as well as one or more pre-conditions that leads to a clinical decision within the decision tree. Some methods for implementing interactive medical guidelines are described in U.S. Patent Publication No. US 2022/0230763 A1, entitled “Method and System for Providing Interactive Medical Guideline,” the entire contents of which is incorporated by reference.

In some embodiments, a patient medical record is mapped to the nodes of an interactive medical guideline, including prior medical decisions and/or preconditions from the patient's record. The interactive guideline may initially include a display of the patient-mapped nodes and alternative branches relative to those nodes. Selecting an alternative branch node, such as by hovering a pointer over the node, may expand the guideline from the selected node, displaying and distinguishing expanded alternative paths of the guideline apart from a patient's currently recorded path. In some embodiments, de-selecting an alternative node, such as by ceasing to hover over a node, collapses the expanded alternative path.

In some embodiments, data from electronic medical records may be dynamically accessed, parsed/identified, and extracted to correlate the data with nodes and paths within a guideline. Parsing and extraction may be implemented by scanning records (e.g., using optical character recognition) and a natural language processor (NLP), after which the processed data is correlated with clinical decisions, tests, diagnosis, and preconditions of the patient and their relationships with each other in accordance with a guideline. In some embodiments, machine learning models are developed for processing medical records and correlating data to elements of a guideline.

In some embodiments, a sequence of mapped or selected nodes and alternate nodes may be associated/stored with a patient medical record and displayed/distinguished accordingly as primary selected nodes and alternate nodes when the corresponding medical guideline and patient record is open for display in the interface. The sequence of clinical decisions selected/added for a patient can be presented as part of treatment history of the patient.

The navigation interface can provide different forms of graphical representation of the medical guideline in different operation modes, such as a tree mode and a step mode. To provide an interactive experience, the navigation interface can also provide different ways of navigating through the medical guideline and selecting clinical decisions in the medical guideline under the different operation modes. The interactive medical guideline engine can also allow the user to store a sequence of clinical decisions selected for a patient, and to add customized treatment/diagnostic tests procedures which are not part of the medical guideline to the sequence.

These and other embodiments of the invention are described in detail below. For example, other embodiments are directed to systems, devices, and computer readable media associated with methods described herein.

A better understanding of the nature and advantages of embodiments of the present invention may be gained with reference to the following detailed description and the accompanying drawings.

### BRIEF DESCRIPTION OF THE DRAWINGS

The detailed description is set forth with reference to the accompanying figures.

FIG. 1A shows an illustrative graphical user interface of an interactive guideline displaying sequences of selected and alternate nodes according to some embodiments.

FIG. 1B shows an illustrative graphical user interface responsive to the selection of an alternate node of FIG. 1A.

FIG. 2 illustrates a system for providing an interactive medical guideline according to some embodiments.

FIG. 3 illustrates a medical guideline data structure according to some embodiments.

FIG. 4A shows an illustrative graphical user interface of an interactive guideline operating in a step mode according to some embodiments.

FIG. 4B illustrates operation of the graphical user interface of FIG. 4A for displaying alternative guideline nodes according to some embodiments.

FIG. 5A shows an illustrative graphical user interface for selecting an interactive guideline according to some embodiments.

FIG. 5B shows an illustrative graphical user interface for the interactive guideline selected from the interface of FIG. 5A according to some embodiments.

FIG. 5C shows the illustrative graphical user interface of FIG. 5B representing a selected sequence of nodes according to some embodiments.

FIGS. 6A and 6B show an illustrative graphical user interface for creating a custom node of an interactive guideline according to some embodiments.

FIG. 7 is an illustrative flow chart for generating and operating a graphical user interface with an interactive guideline according to some embodiments.

FIG. 8 illustrates an example computer system that may be utilized to implement techniques disclosed herein.

### DETAILED DESCRIPTION

Disclosed herein are techniques for providing an interactive medical guideline engine which can address at least some of the issues described above.

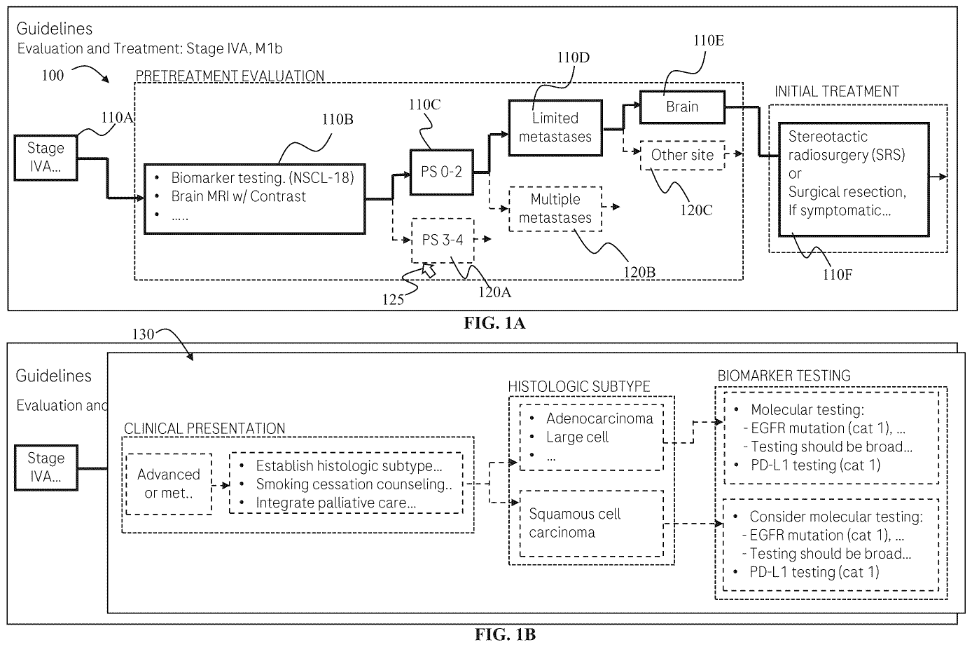

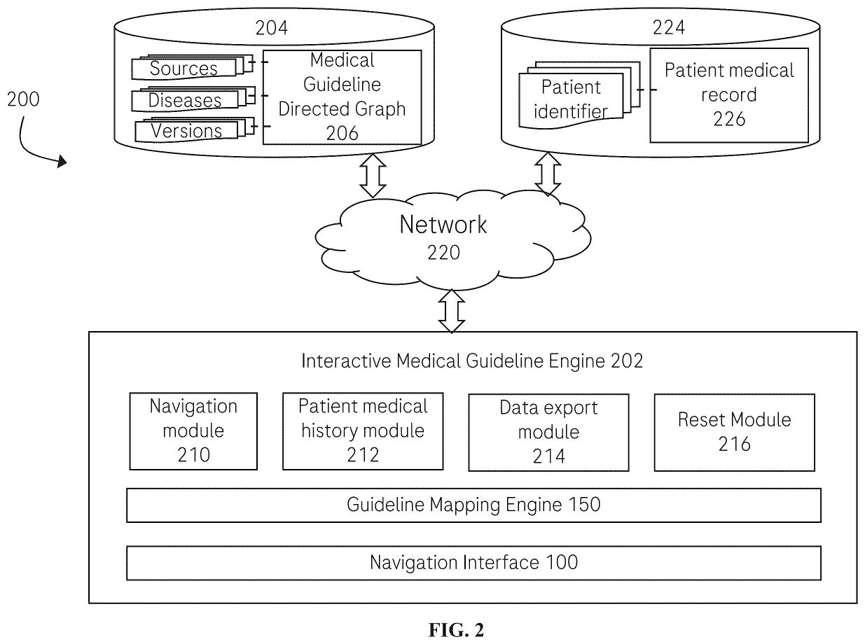

Disclosed FIG. 1A shows an illustrative graphical user interface of an interactive guideline 100 displaying sequences of selected and alternate nodes according to some embodiments. FIG. 1B shows an illustrative graphical user interface responsive to the selection of an alternate node of FIG. 1A. A medical guideline generally represents a decision tree of a plurality of nodes (e.g., representing clinical decisions and preconditions) connected by edges representing dependency relationships among the nodes (e.g., a recommended clinical decision leading from an earlier clinical decision/precondition). A node 110A represents a diagnosis of a patient (e.g., a particular type of cancer) and a node 110B represents recommended and selected diagnostic steps for the patient based on the diagnosis. Node 110C represents a further diagnosis (e.g., performance/functional status) of the patient. Node 110D represents a further diagnosis (e.g., the level of spread/metastasis of cancer) that may be based on the results of the diagnostic steps taken at node 110B (e.g., PET/CT scan). Based on patient medical records, and/or otherwise selected/identified by an operator of the interface, node 110E represents the anatomical location of disease (e.g., cancer of the brain), and node 110F represents the recommended treatment(s) based on the guideline.

Nodes 110A, 110B, and 110C may be selected by an operator through the interactive interface and/or may be dynamically selected based on existing medical records of a patient as further described below. User interface 100 is programmed to detect a user input corresponding to an alternate node 120A from a selection pointer 125. In response to hovering the pointer 125 over node 120A, alternate nodes within a guideline, diverging from the selected or recorded sequence of nodes, are expanded as shown in a pop-up display at 930 of FIG. 1B. In some embodiments, a deselection or cessation of the hovering may cause the display of the one or more further child nodes to be removed from the interface (e.g., by collapsing).

Nodes 120A, 120B, and 120C represent alternative branches of the guideline from those selected/identified as representing a patient's history. In some embodiments, direct child alternative nodes of selected nodes are initially displayed. Alternative nodes may be rendered and visually distinguished/contrasted (e.g., grayed) apart from the other nodes in a different region of a display frame. Alternate node 120A may represent a different diagnosis than node 110D, which is identified as part of the patient's history. For example, an alternate diagnosis may represent an advanced or metastatic level of cancer, as opposed to a limited level of cancer as represented by node 110D. Selecting alternate node 130 (e.g., by hovering a pointer over the node) causes the respective portion of the guideline to be expanded and visible to an operator at 130. The expanded portion of the guideline may be presented in a separate window such as shown in FIG. 1B or otherwise highlighted/differentiated from those portions currently selected/identified with the patient's history.

Nodes of the pop-up display 130 may be displayed, selected, modified, and/or recorded according various embodiments as further described herein. In some embodiments, an input is received that causes selection of the alternate nodes to be associated and included with a medical record of the patient. Annotations/notes from a user may be recorded in reference to the selected alternate nodes.

In some embodiments, no alternative nodes are initially displayed with an interactive medical guideline. In response to receiving particular input (e.g., hovering a cursor over an already selected node), alternative nodes that were not previously selected and shown are displayed and visually distinguished. In response to further input (e.g., ceasing the hovering), the branches of alternative nodes collapse and/or may be selected/stored as part of a patient's medical record.

FIG. 2 illustrates a system for providing an interactive medical guideline according to some embodiments. System 200 includes an interactive medical guideline engine 202, guideline database 224, and medical record database 204 connected over a network 220 (e.g., the internet or a LAN). Database 224 stores data representing multiple medical guideline directed graphs 206 (herein after, “directed graphs 206”), which can be generated from multiple medical guideline documents. The directed graphs can be associated with different publication sources (e.g., NCCN), diseases (e.g., breast cancer, lung cancer, etc.) and versions. Database 204 includes medical records of patients including those pertaining to the conditions and treatments of the guidelines within database 224. Interactive medical guideline engine 202 can include software instructions, which can be executed by hardware processor(s) to perform the various functions described herein.

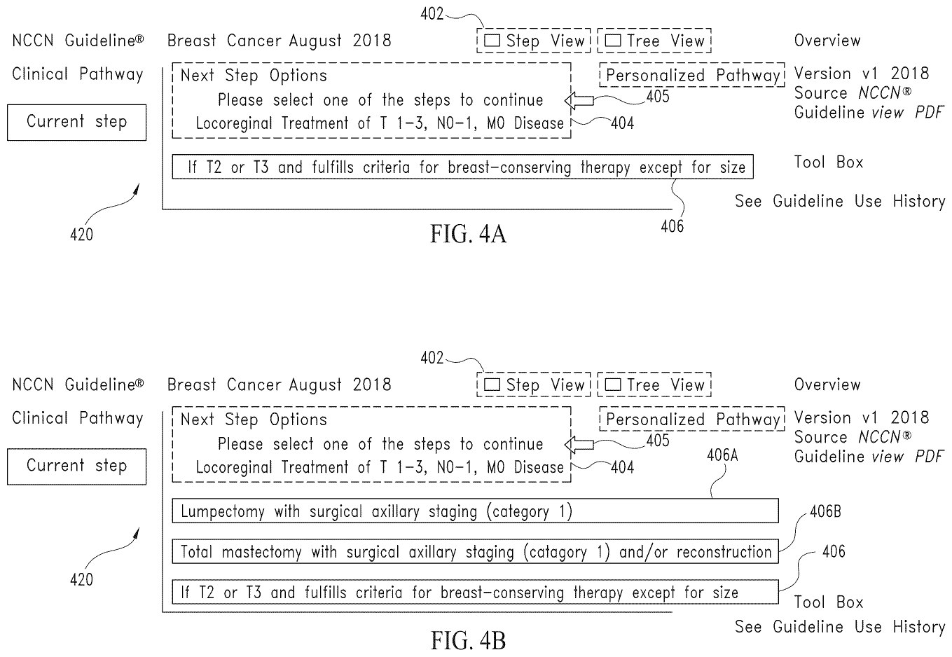

Interactive medical guideline engine 202 can generate a navigation interface utilizing a navigation module 210, to provide a graphical representation of directed graph 206 such as shown and described with respect to FIGS. 1A and 1B. Navigation module 210 can provide different forms of graphical representation of directed graph 206 in different operation modes, such as a tree mode and a step mode (e.g., as shown in FIGS. 4A and 4B). Navigation module 210 is configured to allow a user to navigate through the medical guideline to select/compile a set of clinical decisions.

Medical guideline engine 202 is programmed and configured to access patient medical record database 204 including electronic medical records 226 of patients. In some embodiments, a guideline mapping engine 150 dynamically maps medical records 226 received (and/or transcribed) through patient medical history module 212 for a patient to select nodes of a guideline from database 204, such as nodes 110A, 110B, and 110C illustrated in FIG. 1A. The process may be implemented by scanning records (e.g., using optical character recognition) and a natural language processor (NLP), after which the processed data is correlated with clinical decisions, tests, diagnosis, and preconditions of the patient and their relationships with each other in accordance with a sequence of a guideline. For example, methods of parsing and structuring unstructured medical records are described in U.S. Patent Application Publication No. US 2022/0301670 A1, entitled “Automated Information Extraction and Enrichment in Pathology Report using Natural Language Processing,” by Sharma et al., the entire contents of which is herein incorporated by reference. In some embodiments, machine learning models are developed for processing medical records and correlating data to elements of a guideline.

The interactive medical guideline engine is configured to record the sequence (or path) of selected nodes in the decision tree of the medical guideline. The selected historical nodes and alternative nodes (paths) can be part of the medical record of that patient, and integrated with patient records in database 224 using a data export module 214. As such, the recorded sequence can be stored in a document (e.g., a patient's report, a meeting presentation, etc.) that forms part of the patient's medical record.

Besides the tree mode as shown in FIGS. 1A and 1B, the navigation interface can also operate in a step mode such as shown in FIGS. 4A and 4B. In the step mode, the navigation interface can display the content (e.g., a clinical decision, a pre-condition, etc.) of a currently-selected node of the directed graph, as well as the content of all the direct child nodes of the currently-selected node. In the step mode, the nodes can be represented as boxes, and the edges between the nodes are not displayed. The direct child nodes can represent, for example, all possible options of the next clinical decision, all possible treatment responses/diagnosis results, etc. The step mode allows the user to traverse the directed graph one node at a time. As in the tree mode, the interactive medical guideline engine can also track and record the sequence of user's selection of the nodes in the step mode. In some embodiments, the interactive medical guideline engine can cause the navigation interface to display alternate child nodes, such is in response to a further selection of an already selected parent node (e.g., by hovering a pointer over the node).

In addition, the interactive medical guideline engine can also detect an update to the medical guideline (e.g., from an online source of the guideline) through a reset module 216 and can provide notifications via the navigation interface to the user. The notifications can prompt the user to, for example, remove the sequence of nodes selected from the old medical guideline for a patient, navigate through the new medical guideline to create a new sequence of nodes for the patients, etc., to ensure that the clinician makes the clinical decisions based on the updated medical guideline. In some embodiments, in response to an update, a sequence of nodes is selected dynamically from the updated guideline based on patient records with or without input from a user.

With the disclosed embodiments, a navigation interface allows a user to access different parts of the medical guideline based on simple selection commands (e.g., clicking, zoom in, zoom out, drag, etc.) and providing the graphical representation of at least part of the directed graph including the nodes mapped to the selected region via the navigation interface, with at least a portion of the precondition or clinical decision represented by a node visually represented with the node (e.g., text/graphics), which can substantially simplify the user's access to the medical guideline. By tracking and displaying the sequence of user's selection of nodes in the decision tree of the medical guideline, the user (e.g., a clinician) can keep track of the medical journeys for different patients in view of the medical guideline, which allows the clinician to consult the relevant portions of the medical guideline for different patients to determine their treatments.

Compared with a case where the clinician has to navigate through a substantial guideline document to search for relevant portions of the medical guideline for different patients and alternative diagnosis and treatments, and separately record the guideline steps undertaken by the patients and alternative steps, the disclosed embodiments provide a unified interface to navigate and search the medical guideline and to record the selected steps for different patients. All these can improve the ease of accessing the relevant medical treatment information from the medical guideline for different patients, which in turn can improve the quality of medical treatments provided to those patients.

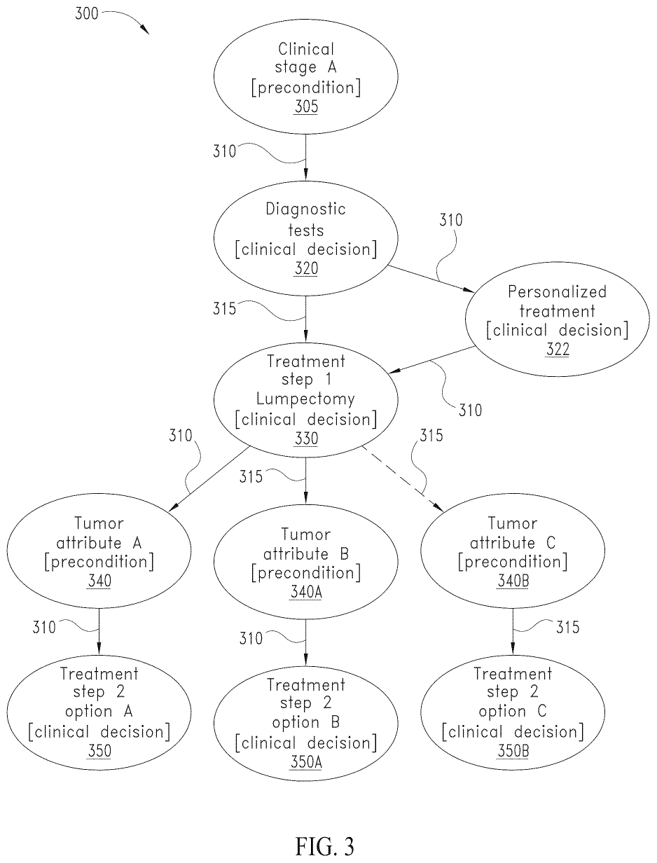

FIG. 3 illustrates a medical guideline data structure according to some embodiments. An instance of the structure may have been originally obtained from guideline database 224, for example. A root node 305 represents a particular precondition of a patient. Node 320 represents a diagnostic test recommended for the patient based on the precondition represented by node 305. Node 322 represents a customized clinical decision node inserted into the medical guideline for a patient (e.g., distinguished from the original guideline structure). Edges 310 represent paths identified/selected for a particular patient while edges 315 represent alternative paths within the guideline.

Node 340 represents a tumor property attributed to a patient based on the type of tumor extracted from a dissection performed at a treatment represented by node 330. Nodes 340A and 340B represent alternative tumor properties of the removed tumor section. Node 350 represents a treatment step recommended and selected for the patient based on the tumor attribute represented by node 340. Nodes 350A and 350B represent alternative treatment nodes differentiated from the selected treatment of node 350 associated with alternative tumor properties of nodes 340A and 340B respectively.

FIG. 4A shows an illustrative graphical user interface of an interactive guideline operating in a step mode according to some embodiments. FIG. 4B illustrates operation of the graphical user interface of FIG. 4A for displaying alternative guideline nodes according to some embodiments. In the step mode, which may be activated in response to a command by an operator from a toggle input 402 (e.g., from the interface of FIG. 1A), a navigation interface 420 displays a particular node 404 (e.g., of a patient's path/history) along with a child node 406 that has also been selected as part of a patient's history. In some embodiments, nodes apart from those that are part of a selected patient path are not initially displayed or are visually distinguished from historical nodes.

As illustrated in FIG. 4B, by hovering a cursor 405 over node 404 (or by another selection mechanism), the remaining child nodes of node 406, nodes 406A and 406B, are displayed and visually distinguished from node 406 to show all child nodes of node 404. In response to a cessation of hovering cursor 405 over node 404 (or by another de-selection mechanism), the other child nodes apart from patient path node 406 are hidden from view. A patient's path within a guideline may be modified in step mode by de-selecting a currently active/selected node of a patient's path and selecting a new node (e.g., node 406A), which then becomes an active/selected node of a patient's path.

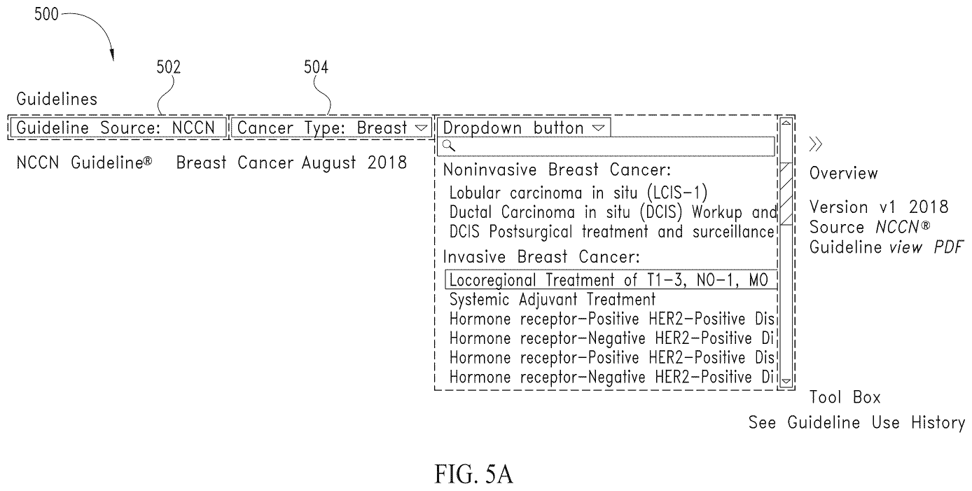

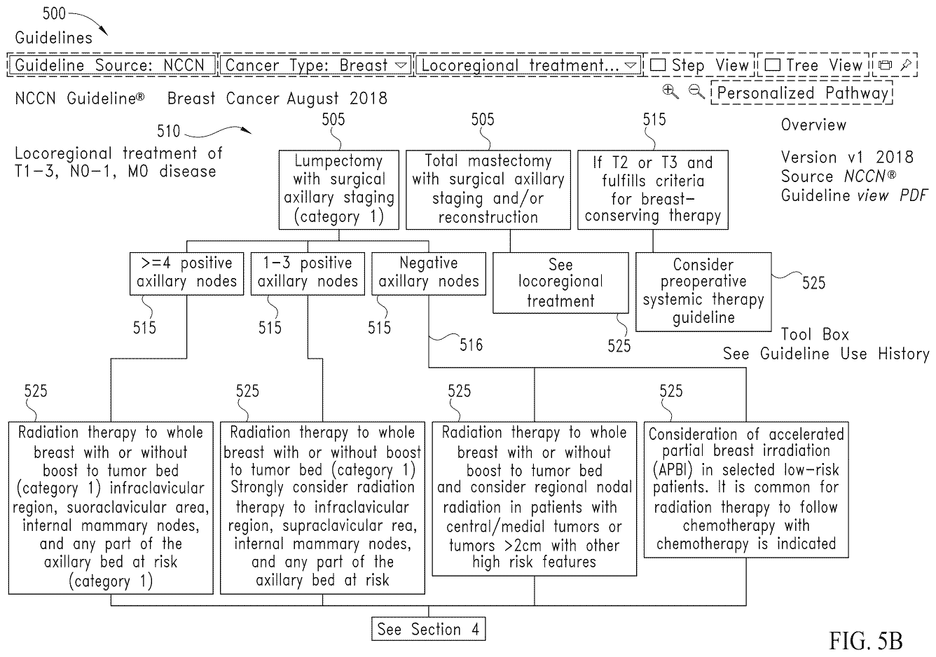

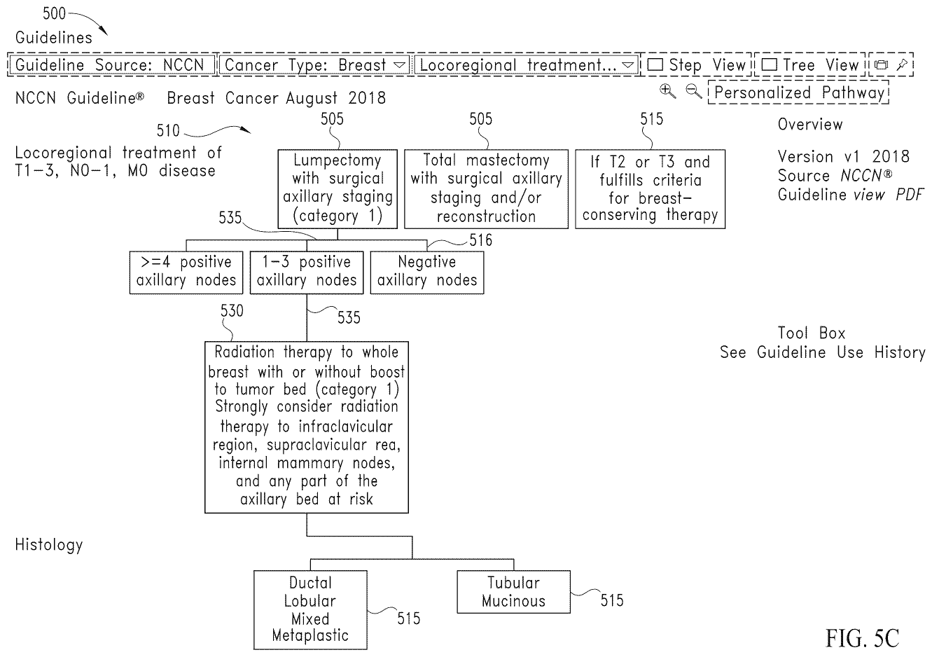

FIG. 5A shows an illustrative graphical user interface for selecting an interactive guideline according to some embodiments. An interface 500 provides menus 502, 504, and 506 for selecting different medical guidelines for interactive interfacing as further described herein. A compilation of guidelines may be obtained from a database (e.g., database 224) such as that stored at a remote repository which is periodically updated. Menus 502, 504, and 506 may be drop-down menus providing categories and sub-categories of various guidelines from which to select. Once selected, a guideline may be dynamically compared with a patient's record, based on which its nodes are selected as further described herein. Such as utilizing interface 100 of FIGS. 1A and 1B, selected/correlated and alternative nodes may be viewed, modified, and made part of a patient's medical record.

A guideline 510 includes nodes 505 representing particular procedures conducted on a patient, nodes 515 representing particular conditions identified for a patient (e.g., based on test results), and nodes 525 representing recommended clinical options for a patient, related to each other with connections 516.

FIG. 5B shows an illustrative graphical user interface 510 for the interactive guideline selected from the interface of FIG. 5A according to some embodiments. The interface includes head nodes 515 representing conditions particular for the guideline with child nodes 525 representing clinical actions (e.g., tests, surgery), further conditions, and/or diagnosis leading to other child nodes.

FIG. 5C shows the illustrative graphical user interface of FIG. 5B representing a selected sequence of nodes according to some embodiments. As described above, sequence of nodes of a guideline may be selected dynamically based on a patient's medical record and/or by interaction of a user. Interface 510 shows selected nodes 530 across selected path connections 535, visually distinguished from non-selected or alternate nodes 505 and 515, etc. As further described with respect to FIGS. 1A and 1B, a user can select alternate nodes and display the expanded portions of the guideline for those alternate nodes, and select the expanded portions to be part of a patient's history for comparison with a patient's (selected) history.

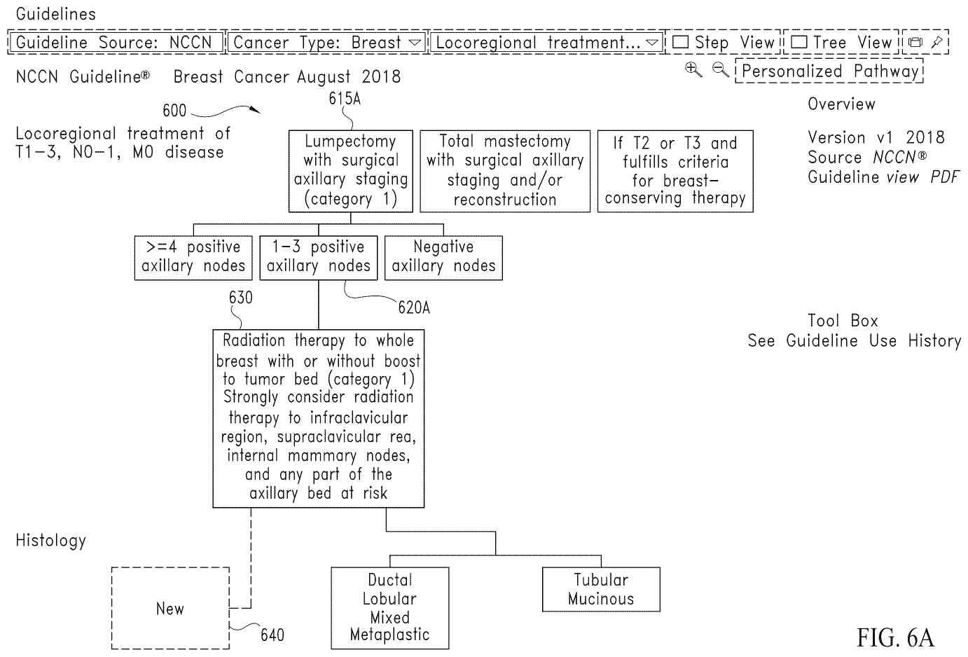

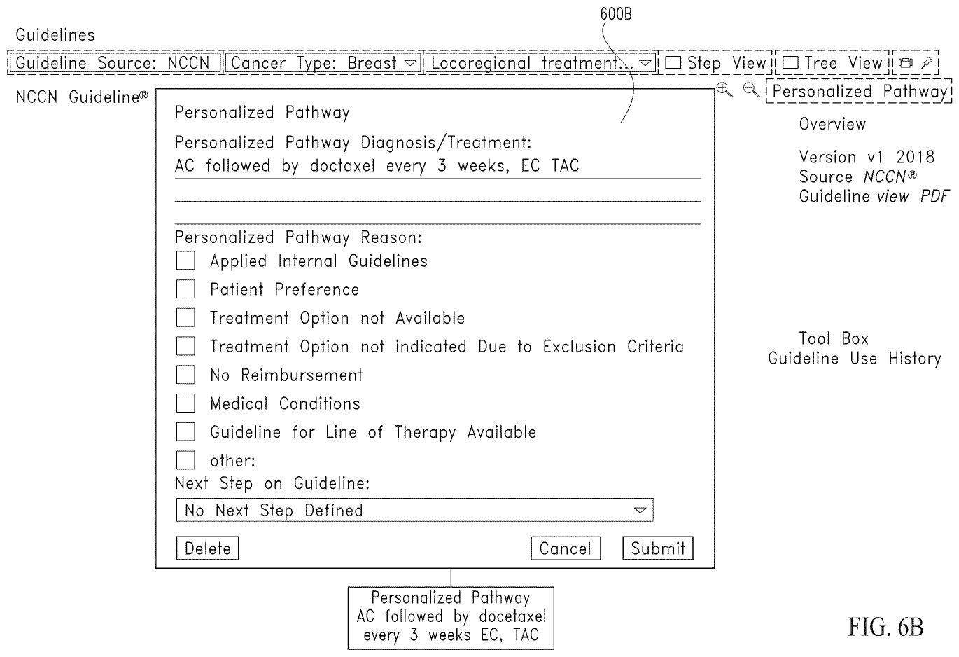

FIGS. 6A and 6B show an illustrative graphical user interface for creating a custom node of an interactive guideline according to some embodiments. An interface 600 provides an interactive interface for a guideline of selected nodes 615A, 620A and 630. In certain circumstances, a patient's history does not entirely align with a guideline. Interface 600 permits a user to generate a custom node 640 (e.g., in response to right-clicking a pointer on node 630). Once a custom node is prompted/requested, interface 600B queries the user for details of the custom node. A user may add details to a custom node including a type of node (e.g., pre-condition, clinical decision-diagnosis, treatment), and a description (e.g., a particular diagnosis, precondition, or treatment). The interface may also prompt the user for the reasons for creating the custom node (e.g., a patient wishes to utilize an experimental treatment not part of the guideline).

In some embodiments, a custom node can be dynamically created based on data/information collected from a patient's medical record (or update to a record), after which a user may be notified of the node's creation that departs from the original guideline. The user may be prompted for confirming/changing details such as may be entered in interface 600B.

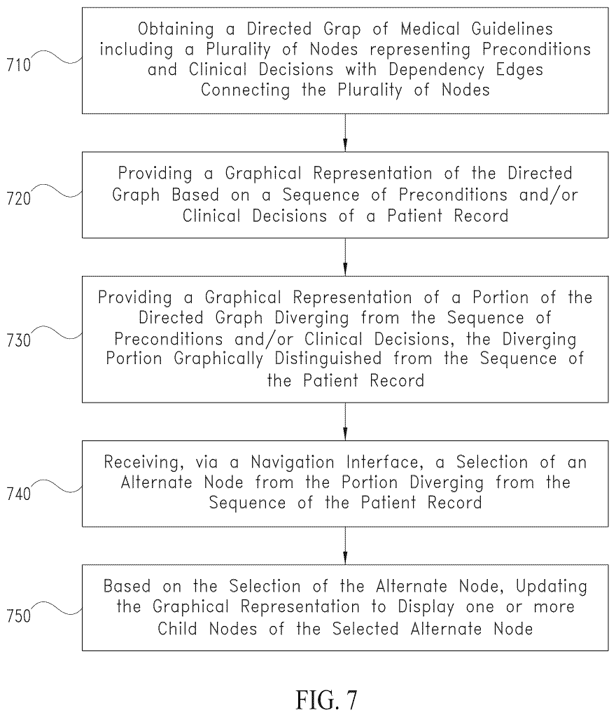

FIG. 7 is an illustrative flow chart for generating and operating a graphical user interface with an interactive guideline according to some embodiments. At block 710, an interactive guideline system/engine obtains a directed graph of medical guidelines (e.g., graph 300 of FIG. 3 from database 224 of FIG. 2). The graph may represent standardized care guidelines and is obtained in digital form (e.g., electronic database records).

At block 720, a patient's medical record is compared with the medical guideline and the record is correlated with paths of the guideline (nodes and edges) such as further described herein. For example, a system may utilize machine learning models to correlate portions of a medical record with medical conditions and clinical decisions that represent paths of the guideline. A user of the interactive guideline may access the interactive guideline to change or add selected nodes. In some embodiments, a user is prompted to verify or complete correlations and/or new nodes.

At block 730, a limited set of alternative nodes/paths of the guideline are displayed together with nodes identified/selected as part of a patient's history, such as shown in FIGS. 1A and 1B. In some embodiments, only selected nodes are shown that are part of a patient's history (e.g., as an initial view), along with guideline paths representing possible future paths of the patient. In some embodiments, direct alternative child nodes of the selected path are displayed. The departing/alternative portions of the guideline may be visually distinguished from nodes representing a patient's history such as for purposes of comparison (e.g., of a different contrast, color, font, size, etc.).

At block 740, input from a user is received that indicates selection of an alternate node (e.g., hovering a cursor over a node for a predetermined amount of time). At block 750, in response to receiving the input from the user, one or more child nodes of the selected alternative path are further displayed (as shown in FIG. 1B).

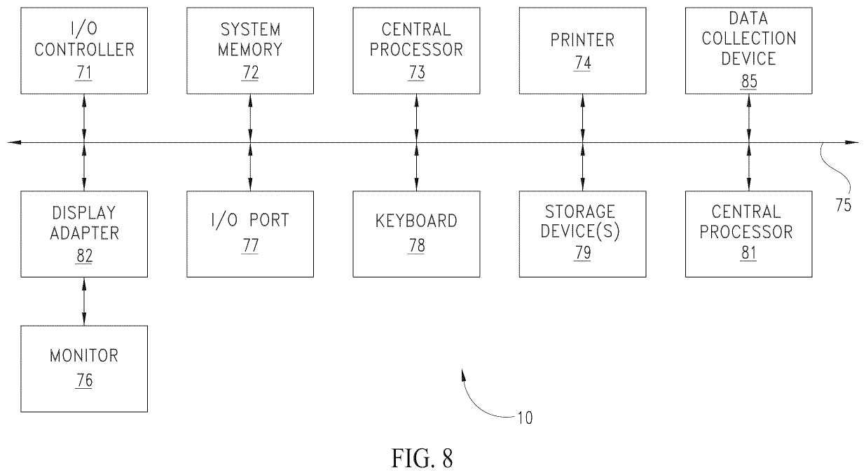

Any of the computer systems mentioned herein, such as for hosting the systems and implementing the processes described herein, may utilize any suitable number of subsystems. Examples of such subsystems are shown in FIG. 8 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. In some embodiments, a cloud infrastructure (e.g., Amazon Web Services), a graphical processing unit (GPU), etc., can be used to implement the disclosed techniques.

The subsystems shown in FIG. 8 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C #, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the invention has been presented for the purposes of illustration and description. It is not intended to be exhaustive or to limit the invention to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover reference to a “first” or a “second” component does not limit the referenced component to a particular location unless expressly stated.

All patents, patent applications, publications, and descriptions mentioned herein are incorporated by reference in their entirety for all purposes. None is admitted to be prior art.

We claim:

1. A computer-implemented method of providing an interactive medical guideline, comprising:
receiving data of a directed graph representing a medical guideline, wherein the medical guideline includes a decision tree including a plurality of clinical decisions and preconditions leading to at least some of the clinical decisions, the directed graph including a plurality of nodes representing the clinical decisions and the preconditions and edges connecting the plurality of nodes to represent dependency relationships among the clinical decisions and the preconditions;
providing, via a navigation interface, a graphical representation of at least part of the directed graph based on a sequence of recorded preconditions and/or clinical decisions;
providing, via the navigation interface, a graphical representation of a portion of the directed graph diverging from the sequence of recorded preconditions and/or clinical decisions, and graphically distinguishing the diverging portion of the directed graph from the portion of the directed graph based on the recorded preconditions and/or clinical decisions;
receiving, via the navigation interface, a selection of an alternate node from the portion of the directed graph diverging from the sequence of recorded clinical decisions and/or preconditions; and
based on the selection of the alternate node, updating the graphical representation of the directed graph to display one or more child nodes of the selected alternate node.

2. The method of claim 1, wherein the selecting comprises hovering a cursor over the alternate node for at least a predetermined period of time.

3. The method of claim 2, wherein a cessation of the hovering over the alternate node causes updating the graphical representation of the directed graph to cease displaying the one or more child nodes of the alternate node.

4. The method of claim 1, wherein the sequence of recorded preconditions and/or clinical decisions is based on an electronic medical record of a patient.

5. The method of claim 4, further comprising receiving, via the navigation interface, a user input and, in response to the user input, associating and storing a record of the alternate node and one or more child nodes of the alternate node with a medical record of the patient.

6. The method of claim 1, wherein graphically distinguishing comprises rendering the selected alternate node and more or more child nodes with a different contrast level or color compared to the part of the directed graph based on the recorded preconditions and/or clinical decisions.

7. The method of claim 1, wherein graphically distinguishing comprises rendering the selected alternate node and one or more child nodes in a pop-up window.

8. The method of claim 1, further comprising:
mapping the plurality of nodes and updated graphical representation of the decision tree to different regions of a display frame;
receiving, via the navigation interface, an input to select a region of the display frame to be displayed in the navigation interface; and
based on the selected region and the mapping, providing the graphical representation of at least part of the directed graph including the nodes mapped to the selected region via the navigation interface.

9. The method of claim 8, wherein the input comprises at least one selected from: a zoom-in command, a zoom-out command, or a drag action.

10. The method of claim 1, wherein each of the nodes are represented with text comprising respective clinical decisions or preconditions.

11. The method of claim 1, further comprising receiving input selecting one or more child nodes of the selected alternate node of the directed graph from the graphical representation and, based on the selection of the one or more child nodes, updating the graphical representation of the at least part of the directed graph to provide a navigation result of the medical guideline representing the selected one or more child nodes of the selected alternate node.

12. The method of claim 1, further comprising:
receiving, via the navigation interface, a request to insert a node representing a customized clinical decision not defined in the medical guideline into the directed graph;
responsive to the request, generating a pop up window to collect information of the customized clinical decision;
generating a customized node including the collected information; and
inserting, based on the collected information, the customized node into the directed graph to generate a customized directed graph.

13. The method of claim 12, wherein the information includes the customized clinical decision to be represented by the customized node, and a node of the directed graph which is to become a direct child node of the customized node.

14. A system having one or more processors configured and programmed to cause the execution of a method comprising:
receiving data of a directed graph representing a medical guideline, wherein the medical guideline includes a decision tree including a plurality of clinical decisions and preconditions leading to at least some of the clinical decisions, the directed graph including a plurality of nodes representing the clinical decisions and the preconditions and edges connecting the plurality of nodes to represent dependency relationships among the clinical decisions and the preconditions;
providing, via a navigation interface, a graphical representation of at least part of the directed graph based on a sequence of recorded preconditions and/or clinical decisions;
providing, via the navigation interface, a graphical representation of a portion of the directed graph diverging from the sequence of recorded preconditions and/or clinical decisions, and graphically distinguishing the diverging portion of the directed graph from the portion of the directed graph based on the recorded preconditions and/or clinical decisions;
receiving, via the navigation interface, a selection of an alternate node from the portion of the directed graph diverging from the sequence of recorded clinical decisions and/or preconditions; and
based on the selection of the alternate node, updating the graphical representation of the directed graph to display one or more child nodes of the selected alternate node.

15. The system of claim 14, wherein the selecting comprises hovering a cursor over the alternate node for at least a predetermined period of time.

16. The system of claim 15, wherein a cessation of the hovering over the alternate node causes updating the graphical representation of the directed graph to cease displaying the one or more child nodes of the alternate node.

17. The system of claim 14, wherein the sequence of recorded preconditions and/or clinical decisions is based on an electronic medical record of a patient.

18. A non-transient computer readable medium with instructions for causing one or more processors to execute a method comprising:
receiving data of a directed graph representing a medical guideline, wherein the medical guideline includes a decision tree including a plurality of clinical decisions and preconditions leading to at least some of the clinical decisions, the directed graph including a plurality of nodes representing the clinical decisions and the preconditions and edges connecting the plurality of nodes to represent dependency relationships among the clinical decisions and the preconditions;
providing, via a navigation interface, a graphical representation of at least part of the directed graph based on a sequence of recorded preconditions and/or clinical decisions;
providing, via the navigation interface, a graphical representation of a portion of the directed graph diverging from the sequence of recorded preconditions and/or clinical decisions, and graphically distinguishing the diverging portion of the directed graph from the portion of the directed graph based on the recorded preconditions and/or clinical decisions;
receiving, via the navigation interface, a selection of an alternate node from the portion of the directed graph diverging from the sequence of recorded clinical decisions and/or preconditions; and
based on the selection of the alternate node, updating the graphical representation of the directed graph to display one or more child nodes of the selected alternate node.

19. The non-transient computer readable medium of claim 18, wherein graphically distinguishing comprises rendering the selected alternate node and one or more child nodes in a pop-up window.

20. The non-transient computer readable medium of claim 18, the method further comprising:
mapping the plurality of nodes and updated graphical representation of the decision tree to different regions of a display frame;
receiving, via the navigation interface, an input to select a region of the display frame to be displayed in the navigation interface; and
based on the selected region and the mapping, providing the graphical representation of at least part of the directed graph including the nodes mapped to the selected region via the navigation interface.
